(19) United States
(12) Patent Application Publication
(10) Pub. No.: US 2016/0184441 A1
(43) Pub. Date: Jun. 30, 2016

(54) PROLAMIN-BASED FILM AND METHOD FOR MANUFACTURING PROLAMIN-BASED ORAL FILM FOR PROTECTING ACTIVITY OF ANTI-OXIDATIVE MATERIAL

(71) Applicant: SEHABIO CO., LTD., lncheon (KR)

(72) Inventors: Nam Hoon CHO, lncheon (KR); Sung Moon LEE, Seoul (KR)

(21) Appl. No.: 15/066,092

(22) Filed: Mar. 10, 2016

Related U.S. Application Data
(63) Continuation of application No. PCT/KR2015/001414, filed on Feb. 12, 2015.

(30) Foreign Application Priority Data
Mar. 31, 2014 (KR) 10-2014-0038163

(51) Int. Cl. A61K 47/42; A61K 38/45; A61K 47/22; A61K 31/355; A61K 9/70; A61K 9/00; A61K 38/44; A61K 31/375

(52) U.S. Cl. CPC A61K 47/42; A61K 38/446; A61K 38/44; A61K 38/45; A61K 31/375; A61K 31/355; A61K 9/7007; A61K 9/0053; A61K 47/22

(57) ABSTRACT

Exemplary embodiments relate to a oral film including an anti-oxidative material having a high level of activity and a method for manufacturing the oral film for protecting the activity of an anti-oxidative material. The activity of the anti-oxidative material is retained under harsh conditions. An exemplary embodiment includes manufacturing a prolamin-based film for oral delivery. The method includes preparing a solution containing prolamin, glycerol formal, an anti-oxidative material, and a solvent including alcohol, and evaporating the solvent under a reduced pressure condition at a temperature of 0° C. to 80° C. An exemplary embodiment also includes a film for oral delivery, prepared by the preceding method, the film includes an anti-oxidative material that maintains activity at a high level. An exemplary embodiment also includes a film for oral delivery, prepared by the immediately preceding method, the film including prolamin, glycerol formal, and an anti-oxidative material having a high level of activity.

### CROSS-REFERENCE TO RELATED APPLICATIONS

This application is a continuation of International Patent Application No. PCT/KR2015/001414, filed on Feb. 12, 2015, and claims priority from and the benefit of Korean Patent Application No. 10-2014-0038163 filed on Mar. 31, 2014, each of which is hereby incorporated by reference for all purposes as if fully set forth herein.

### BACKGROUND

1. Field

Exemplary embodiments relate to a prolamin-based oral film and a method for manufacturing a prolamin-based oral film for protecting the activity of an anti-oxidative material. In particular, the present disclosure relates to a prolamin-based oral film including and an anti-oxidative material as well as a method for manufacturing a prolamin-based film for oral delivery, the method including the steps of (a) preparing an aqueous alcohol solution containing prolamin, glycerol formal and an anti-oxidative material; and (b) evaporating a solvent under a reduced pressure condition at a temperature of 0° C. to 80° C.

2. Discussion of the Background

A great amount of reactive oxygen species are produced during an oxidation process for the generation of energy in the body. While being mostly removed by corresponding removal mechanisms in the body, these reactive oxygen species may cause a variety of diseases in the case where a large amount of reactive oxygen species are produced in an instant or chronically, leading to imbalance with anti-oxidative systems in the body. During their metabolism, some oxygen is converted into oxygen free radicals such as superoxide radical (O2—.), hydrogen peroxide (H2O2), hydroxyl radical (HO.), and singlet oxygen (1O2). These oxygen free radicals destroy a balance with anti-oxidative materials composed of anti-oxidative enzymes and non-enzymatic anti-oxidative materials, causing damage to parts of the body.

Free radicals or reactive oxygen species (ROS) are considered to be one of major causes for various diseases or disorders such as aging, cancer, cerebrovascular disease, and cardiovascular disease. Reactive oxygen species may refer to highly reactive oxygens required for the maintenance of an organism in response to various stresses including biological stresses due to pathogens invading an organism or environmental stresses due to the deterioration of the environment around the globe. These highly reactive oxygens may cause serious physiological disorders. More specifically, free radicals or reactive oxygen species have been reported to destroy cells, cut off or induce cross-linking among the connective tissues in the dermal layers of the skin, resulting in dermatological disorders such as the formation of wrinkle, atopic dermatitis, acne and skin cancer; cardiovascular disorders such as cancer, myocardial infarction, stroke, and atherosclerosis; and chronic degenerative diseases such as amyotrophic lateral sclerosis, Alzheimer's disease (AD) and Parkinson's disease (PD). In addition, free radicals or reactive oxygen species are able to produce harmful substances called lipid peroxides by lipid peroxidation in the body. It has been reported that lipid peroxides act on blood vessels, leading to various adult diseases and chronic disorders such as atherosclerosis and blood clots.

As described above, since free radicals or reactive oxygen species are associated with various disorders, there have been continuous efforts in preventing or treating diseases by administering a diversity of anti-oxidative materials which inhibit the formation of reactive oxygen species or rapidly remove those already formed in the body.

Regarding treatment of diseases, especially chronic diseases, the most typical and convenient mode of drug administration is performed via oral route. However, when administered orally, such anti-oxidative materials suffer from a loss or reduction in their biological efficacy while moving through harsh conditions (such as pH change and various digestive enzymes) of the gastrointestinal tract including the stomach and the small intestine, significantly limiting their effectiveness in vivo. Especially, when administered orally per se, therapeutic proteins are known to be easily degradable in the environment of gastrointestinal tract (See Woodley, J. F., Enzymatic Barriers for GI Peptide and Protein Delivery, CRIT. REV. THER. DRUG CARRIER SYST. 11, 61-5 (1994)).

Hence, a new strategy is necessary for enhancing an effect of oral drug delivery system in which the activity of an anti-oxidative material is highly protected from a digestive process in the stomach and the small intestine.

The above information disclosed in this Background section is only for enhancement of understanding of the background of the invention and therefore it may contain information that does not form any part of the prior art nor what the prior art may suggest to a person of ordinary skill in the art.

### SUMMARY

Additional features of the invention will be set forth in the description which follows, and in part will be apparent from the description, or may be learned by practice of the invention.

While studying on an oral drug delivery system effective for protecting the activity of an anti-oxidative material from digestive organs, the present inventors have confirmed that the film for oral delivery as prepared by the various exemplary embodiments maintains the activity of an anti-oxidative material at a significantly high level.

An exemplary embodiment discloses a method for preparing a film for oral delivery, the method including (a) preparing a solution containing prolamin, glycerol formal an anti-oxidative material, and a solvent including alcohol, and (b) evaporating the solvent under a reduced pressure condition at a temperature of 0° C. to 80° C.

An exemplary embodiment also discloses a prolamin-based film for oral delivery containing an anti-oxidative material which is prepared by the method described above.

An exemplary embodiment further discloses a film including prolamin, glycerol formol, and an anti-oxidative material having a high level of activity.

The foregoing general description and the following detailed description are exemplary and explanatory and are intended to provide further explanation of the claimed subject matter.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the principles of the invention.

FIG. 1 is a diagram showing a SOD activity in each test group at pH 7.0 (Zein: Zein film, SOD: free superoxide dismutase, Zein-SOD: Zein-based film for oral delivery containing SOD).

FIG. 2 is a diagram showing a relative activity of catalase in each test group at pH 7.0 (Zein: Zein film, CAT: free catalase, Zein-CAT: Zein-based film for oral delivery containing catalase).

FIG. 3 is a diagram showing a relative activity of catalase in each test group at pH 7.0 (Gliadin: Gliadin film, CAT: free catalase, Gliadin-CAT: Gliadin-based film for oral delivery containing catalase).

FIG. 4 is a diagram showing a SOD activity in each test group at pH 1.3 which is the condition similar to that of the stomach (White graph: Pepsin-treated group, Black graph: Pepsin-untreated group, Zein: Zein film, SOD: free superoxide dismutase, Zein-SOD: Zein-based film for oral delivery containing SOD).

FIG. 5 is a diagram showing a relative activity of catalase in each test group at pH 1.3 which is the condition similar to that of the stomach (White graph: Pepsin-treated group, Black graph: Pepsin-untreated group, Zein: Zein film, CAT: free catalase, Zein-CAT: Zein-based film for oral delivery containing catalase).

FIG. 6 is a diagram showing a relative activity of catalase in each test group at pH 1.3 which is the condition similar to that of the stomach (White graph: Pepsin-treated group, Black graph: Pepsin-untreated group, Gliadin: Gliadin film, CAT: free catalase, Gliadin-CAT: Gliadin-based film for oral delivery containing catalase).

FIG. 7 is a diagram showing a SOD activity in each test group at pH 7.4 which is the condition similar to that of the small intestine (White graph: Trypsin-treated group, Black graph: Trypsin-untreated group, Zein: Zein film, SOD: free superoxide dismutase, Zein-SOD: Zein-based film for oral delivery containing SOD).

FIG. 8 is a diagram showing a relative activity of catalase in each test group at pH 7.4 which is the condition similar to that of the small intestine (White graph: Trypsin-treated group, Black graph: Trypsin-untreated group, Zein: Zein film, CAT: free catalase, Zein-CAT: Zein-based film for oral delivery containing catalase).

FIG. 9 is a diagram showing a relative activity of catalase in each test group at pH 7.4 which is the condition similar to that of the small intestine (White graph: Trypsin-treated group, Black graph: Trypsin-untreated group, Gliadin: Gliadin film, CAT: free catalase, Gliadin-CAT: Gliadin-based film for oral delivery containing catalase).

FIG. 10 is a diagram showing a relative activity of catalase which is contained in the conventional nanoparticle formulation and the prolamin-based film for oral delivery according to an exemplary embodiment, respectively.

FIG. 11 is a diagram showing a relative activity of catalase which is contained in films for oral delivery prepared by the conventional oral film preparation method and the prolamin-based oral film preparation method according to an exemplary embodiment, respectively.

FIG. 12 is a diagram showing a relative catalase activity depending on varying temperatures of 2° C., 15° C., 25° C., 35° C., 45° C., 55° C., 65° C., and 75° C. during the preparation of the prolamin-based film for oral delivery according to an exemplary embodiment.

FIG. 13 is a diagram showing a relative catalase activity at the temperature of 0° C. or 15° C. during the preparation of the prolamin-based film for oral delivery according to an exemplary embodiment.

FIG. 14 is a diagram showing a relative catalase activity depending on varying pressures during the preparation of the prolamin-based film for oral delivery according an exemplary embodiment.

FIG. 15 is a diagram showing an anti-oxidative activity at varying pH conditions during the preparation of prolamin (either Zein or Gliadin)-based film for oral delivery containing a non-enzymatic anti-oxidative material, i.e., caffeine.

### DETAILED DESCRIPTION

In the following description, for the purposes of explanation, numerous specific details are set forth in order to provide a thorough understanding of various exemplary embodiments. It is apparent, however, that various exemplary embodiments may be practiced without these specific details or with one or more equivalent arrangements. In other instances, well-known structures and devices are shown in block diagram form in order to avoid unnecessarily obscuring various exemplary embodiments.

For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. Like numbers refer to like elements throughout. As used herein, the term “and/or” includes any and all combinations of one or more of the associated listed items.

Although the terms “first,” “second,” etc. may be used herein to describe various elements, these elements, should not be limited by these terms. These terms are used to distinguish one element from another element. Thus, a first element, discussed below could be termed a second element without departing from the teachings of the present disclosure.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “contains,” “containing,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and will not be interpreted in an idealized or overly formal sense, unless expressly so defined herein.

Hereinafter, the exemplary embodiments will be described in more detail.

An exemplary embodiment includes a method for preparing a film for oral delivery, the method comprising the steps of (a) preparing an aqueous alcohol solution containing prolamin, glycerol formal and an anti-oxidative material; and (b) evaporating a solvent under a reduced pressure condition at a temperature of 0° C. to 80° C.

In step (a), an aqueous alcohol solution containing prolamin, glycerol formal and an anti-oxidative material is prepared.

Prolamins are a group of spherical proteins which are found in plants such as cereal grains. Prolamin proteins are generally dissolved in 70-80% alcohol solutions, whereas being insoluble in water or pure alcohol. These proteins contain high contents of glutamic acid and proline.

The term prolamin, as used herein, includes any known type of prolamin proteins derived from a plant source. For instance, prolamin may be at least one prolamin derived from a plant source selected from the group consisting of wheat, rye, barley, oat, rice, sorghum, millet, coix and maize. Preferably, prolamin may be one or a mixture of two or more selected from the group consisting of Zein, gliadin, hordein, secalin, kafirin, coixin, glutenin, GluB-Glutelin and avenin.

Glycerol formal (C₄H₈O₃) is also referred to as various name including Methylidinoglycerol; Sericosol N; 1,3-Dioxolane-4-methanol; 1,2-(Methylidene)glycerol; a mixture of 5-hydroxy-1,3-dioxane and 4-hydroxymethyl-1,3-dioxane; Glycerin formal; and Formal Glycerol. Glycerol formal (C₄H₈O₃) is known as a solubilizer for dissolving water-insoluble chemical compounds.

Glycerol formal as used herein has a chemical structure of the following Chemical Formula 1 or 2, and is a mixture of 47-67% of 5-hydroxy-1,3-dioxane basis and 33-53% of 4-hydroxymethyl-1,3-dioxane basis. Glycerol formal as used herein may be commercially purchased or prepared by chemical synthesis methods known in the art.

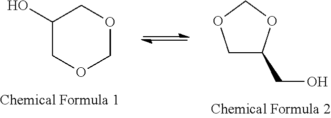 [SMILES: OC1COCOC1 ; OC[C@H]1COCO1]

The term “anti-oxidation,” as used herein, means to prevent various oxidation occurring in the body, while “anti-oxidative material (or antioxidant),” as used herein, refers to a substance which delays or inhibits the oxidation of its substrate material with its relatively smaller amount than its substrate material. “An anti-oxidative material,” as used herein, includes, but is not limited to, any material which possesses a capability (i.e., an anti-oxidative activity) of removing free radicals or reactive oxygen species (ROS) or inhibiting their production, and any material which has an ability (i.e., an anti-oxidation enhancing activity) of promoting such an anti-oxidative activity. In particular, anti-oxidative material includes an anti-oxidative enzyme and a non-enzymatic anti-oxidative material.

An anti-oxidative enzyme refers to a protein possessing the activity of anti-oxidation or enhancing anti-oxidation. It includes any proteins in the art which has been known for their involvement with an anti-oxidative system in the body. Preferably, it may be at least one selected from the group consisting of superoxide dismutase, catalase, glutathione peroxidase, glutathione reductase, glutathione-S-transferase hemeoxygenase, a synthetic enzyme thereof, and a peptide mimetic thereof.

The non-enzymatic anti-oxidative material refers to a chemical compound possessing the activity of anti-oxidation or enhancing anti-oxidation. It includes any chemical compound in the art which has been known for their involvement with an anti-oxidative system in the body. Preferably, it may be at least one selected from the group consisting of vitamin E, curcumin, carotene, lipoic acid, Coenzyme Q10, tocopherol, polyphenol, retinyl palmitate, thiotic acid, carotenoid, vitamin C, vitamin C derivatives, caffeine, caffeic acid, butylated hydroxyanisole (BHA), butylated hydroxytoluene (BHT), tertiary butylhydroquinone (TBHQ), epigallocatechin-3-gallate (EGCG), proanthocyanidin, Gallic acid, selenium, zinc, taurine, flavonoid, and scopoletin.

The solvent used in step (a) according to an exemplary embodiment includes any material which is able to dissolve both prolamin and glycerol formal. Preferably, the solvent may be alcohol.

“Alcohol,” as used herein, may be preferably lower alcohol of C1-C6, most preferably ethanol. Ethanol is a volatile, flammable, colorless liquid which is a 2-carbon alcohol derived from ethane of which a hydrogen atom is replaced by a hydroxyl group.

Alcohol, as used herein, may be utilized as an aqueous alcohol solution by mixing and diluting 100% alcohol with a certain volume ratio (v/v) of water. Thus, the concentration of alcohol as used herein is indicated as a volume ratio of alcohol to the total volume of the solution. “Water” as used herein may be distilled water or deionized water. The amount of water to be added may be 40% (v/v) to 95% (v/v), preferably 55% (v/v) to 75% (v/v), most preferably 70% (v/v), without being limited thereto.

The step (a) of preparing “an aqueous alcohol solution containing prolamin, glycerol formal, and an anti-oxidative material” may be sequentially performed by specifically including, without being limited thereto,

(i) dissolving prolamin in an aqueous alcohol solution;

(ii) adding glycerol formal to the aqueous alcohol solution of step (i); and

(iii) adding an anti-oxidative material to the solution of step (ii).

The ratio of prolamin to an anti-oxidative material may be, without being limited thereto, preferably a weight ratio of 1-1,000,000 to 1, more preferably 1-1000 to 1, most preferably 100-600 to 1.

The ratio of prolamin to glycerol formal may be, without being limited thereto, preferably a weight ratio of 1-10,000 to 1, more preferably 1-100 to 1, most preferably 1-10 to 1.

In the step (b), the aqueous alcohol solution as prepared in the step (a) containing prolamin, glycerol formal and an anti-oxidative material is placed under a reduced pressure at a temperature of 0° C. to 80° C. so as to render said components of the aqueous alcohol solution continuously react with another, leading to the evaporation of a solvent and thereby the formation of a film.

The temperature during evaporation may be, without being limited thereto, specifically in the range of 0° C. to 80° C., preferably in the range of 0° C. to 45° C., more preferably in the range of 10° C. to 38° C., most preferably in the range of 15° C. to 35° C.

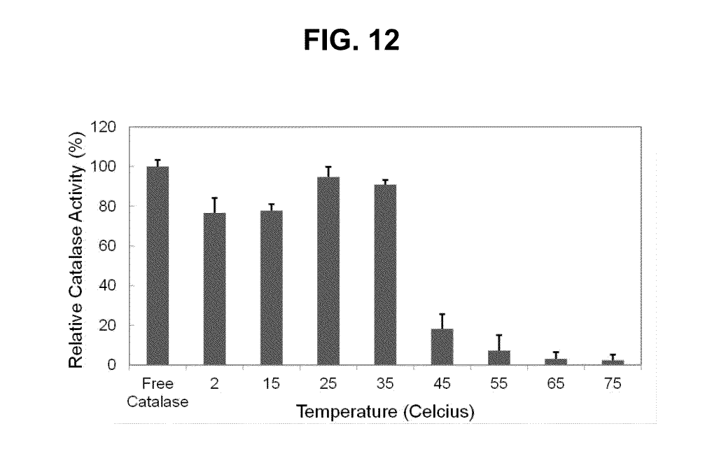

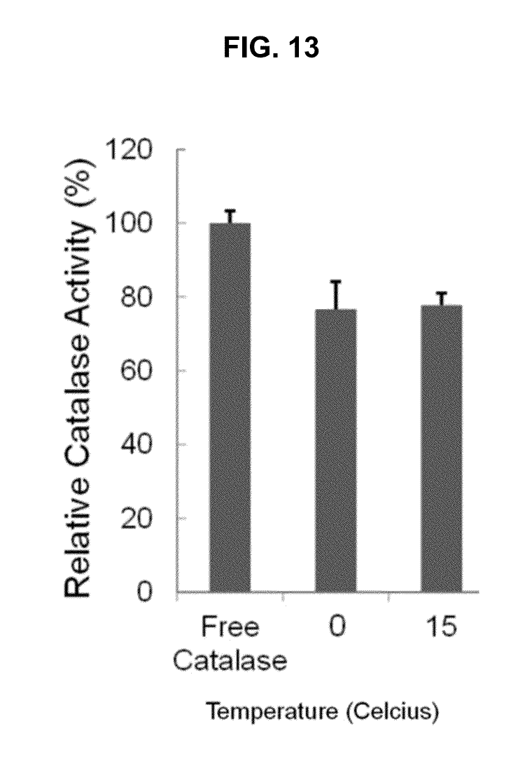

In one Example, the relative activities of the anti-oxidative enzyme contained inside the film for oral delivery prepared according to an exemplary embodiment was measured at varying temperatures under the same pressure. As shown in FIG. 12, catalases contained in the films for oral delivery prepared at 15° C., 25° C., 35° C. and so on maintained their relative activities at a significantly high level, respectively, while catalase contained in the film for oral delivery prepared at 45° C. kept its relative activity 20% and up to 30%. In addition, FIG. 13 demonstrates that catalase contained in the film for oral delivery prepared at 0° C. possessed its high relative activity.

The evaporation of the solvent is conducted under a reduced pressure which may be preferably in the range of 1×10⁻⁵ mbar to 999 mbar, more preferably in the range of 1×10⁻⁴ mbar to 1×10⁻² mbar.

The temperature and the pressure may be maintained for an appropriate time period during which the solvent is sufficiently evaporated. The time period may be varied depending on the amount and the type of a solvent used. Without being limited thereto, the time during which the conditions of the temperature and the pressure are maintained may be preferably 10 seconds to 12 hours, more preferably 1 minute to 1 hour.

In accordance with the preparation method of the various exemplary embodiments including steps (a) and (b), an film for oral delivery maintaining the activity of an anti-oxidative material contained therein at a high level may be prepared.

In particular, the film for oral delivery retained the activity of an anti-oxidative material contained therein at a high level even in the harsh conditions of the stomach and the small intestine (See Example 2).

Further, in case where the same amount of Zein and an anti-oxidative material are used, the film for oral delivery prepared according to an exemplary embodiment contains much larger amount of an anti-oxidative material and thus retains the activity of an anti-oxidative material effectively, in comparison with the nanoparticle prepared by the conventional method (See Experimental Example 1).

Hence, an exemplary embodiment provides a prolamin-based film for oral delivery containing an anti-oxidative material that protects the activity of the anti-oxidative material.

The film for oral delivery according to an exemplary embodiment is prepared by a method including the steps (a) and (b) which were described above.

The amount of an anti-oxidative material contained in the film for oral delivery according various exemplary embodiments may be selected appropriately by one skilled in the art based on the type, the width and the surface area of the film. The film according to various exemplary embodiments for oral administration is capable of containing various amounts of an anti-oxidative material, preferably 0.01 to 80% (w/w), more preferably 0.1 to 10% (w/w).

The film for oral delivery according to various exemplary embodiments includes an anti-oxidative material as an active pharmaceutical ingredient and is able to orally administer the pharmaceutical ingredient simply and rapidly to a subject. In addition, while being prepared on the basis of prolamin under specific conditions, the film according to various exemplary embodiments contains an anti-oxidative material which maintains a high level of activity. In particular, the physiological activity of an anti-oxidative material may be retained at a high level even under the harsh conditions of the gastrointestinal tract.

In terms of the period of drug release, the film for oral delivery according to various exemplary embodiments may be formulated in the form of immediate and/or modified release, preferably an immediately disintegrating tablet or an orally disintegrating film.

Except for prolamin, glycerol formal, and an anti-oxidative material, the film for oral delivery according to various exemplary embodiments may further comprise an appropriate amount of additives depending on various purposes. For instance, the additives include fillers, plasticizers, acidifiers, anti-aggregating agents, solubilizers, stabilizers, sweeteners, corrigents, emulsifiers, various dissolution auxiliary agents, pigmenting agents, flavoring agents and so on. Especially, while the film is prepared according to various exemplary embodiments, film-forming substrates, viscosity enhancers, emulsifiers, starches and the like may be further added.

The film-forming substrates may include any material, as long as it provides the film with flexibility, facilitates the disintegration of the film, and is equipped with its ability of maintaining a viscosity for the formation of particles. Preferably, it may include, without being limited thereto, a polymer such as hypromellose, hydroxypropylcellulose, polyvinylalcohol, polyethyleneoxide and polyvinylpyrollidone. Further, a viscosity enhancer may include pullulan, starch, lactic acid, cellulose, carrageenan, arabic gum, guar gum, locust bean gum, xanthan gum, gellan gum, agar, alginic acid, sodium alginate and so on.

The fillers act to reduce the slippery property of the film in the oral cavity and provide a framework for the film. In addition, it decreases an adhesion among the films, while controlling the stickiness of the film and the rates of film dissolution and drug elution in the oral cavity. For instance, the fillers may include microcrystalline cellulose, cellulose polymer, magnesium carbonate, calcium carbonate, limestone powder, silicate, clay, talc, titanium dioxide, and calcium phosphate.

The plasticizers, which provide a flexibility for the film, may include triethyl citrate, glycerin esters of fatty acid, polyethylene glycol, sorbitol, maltitol, xylitol, glycerin, and so on.

The acidifiers may include citric acid, malic acid, fumaric acid, tartaric acid, ascorbic acid, succinic acid, adipic acid and lactic acid. The anti-aggregating agents may include talc and calcium stearate.

The solubilizers may include sucrose fatty acid ester, sorbitan monostearate, and sodium lauryl sulfate. The stabilizers may include light anhydrous silicic acid, magnesium aluminate meta-silicic acid, magnesium silicate, aluminium silicate, and calcium silicate.

Sweeteners may include aspartame, saccharin, glycyrrhizin potassium, and stevia. Corrigents may include 1-menthol, sodium chloride, acesulfame potassium, and sucralose. Emulsifiers may include glycerin fatty acid ester, sucrose fatty acid ester, lecithin, enzyme-disintegrated lecithin, polysorbate, sorbitan fatty acid ester and propylene glycol. Dissolution auxiliary agents may include cyclodextrin, arginine, lysine, and trisaminomethane.

Further, the film for oral delivery according to various exemplary embodiments additionally includes an appropriate amount of pigmenting agents for the purpose of obtaining a desired color. For instance, the pigmenting agent may include a natural colorant and an edible dye which are commonly known as FD&C dyes.

The film for oral delivery according to various exemplary embodiments may be used for preventing and treating various tissue damages or disorders caused by diverse oxidative reactions of free radicals or reactive oxygens in the body. Various tissue damages or disorders caused by diverse oxidative reactions of free radicals or reactive oxygens in the body are well known in the art and may include, without being limited thereto, inflammation, infection, artherosclerosis, hypertension, cancer, radiation-induced damage, neurological disorder, neurodegenerative disorder, ischemia/reperfusion damage, aging, wounds healing, glutathione deficiency, AIDS, sickle cell anemia, diabetes and rheumatoid arthritis.

An exemplary embodiment provides a method for manufacturing a film for oral delivery, the method including: (a) preparing an aqueous alcohol solution containing prolamin, glycerol formal, and an anti-oxidative material, and (b) evaporating a solvent under a reduced pressure condition at a temperature of 0° C. to 80° C. An exemplary embodiment also provides a prolamin-based film for oral delivery containing an anti-oxidative material as prepared by the immediately preceding method. An exemplary embodiment also provides a film including prolamin, glycerol formol, and an anti-oxidative material having a high level of activity. In accordance with the various exemplary embodiments, a film including an anti-oxidative material that maintains a high level of activity is prepared for oral delivery. The activity of an anti-oxidative material as contained in the film may be retained at a high level even under the harsh conditions of the gastrointestinal tract.

Hereinafter, various exemplary embodiments will be described in detail with reference to the following examples. However, the following examples are merely for illustrating various exemplary embodiments and are not intended to limit the scope of any exemplary embodiment.

### Comparative Example 1

### Preparation of Nanoparticle Containing an Anti-Oxidative Enzyme

### Comparative Example 1-1

100 mg of Zein (Sigma-Aldrich) was dissolved in 1.825 ml of 70% aqueous alcohol solution. 52.5 mg of Sorbitol (Sigma-Aldrich) and 67.5 mg of glycerol formal (Sigma-Aldrich) were added to the solution and mixed until the solution became clear. In order to capsulate an anti-oxidative material with Zein, 3 mg/ml of catalase (EC 1.11.16.1, Sigma-Aldrich) was added dropwise to the solution and stirred to obtain a mixed solution (A total of 200 μl of catalase was added). A nanoparticle was prepared by using a phase separation method. Shortly thereafter, 8.0 ml of water was added to the above prepared mixed solution which was then dispersed by homogenization at 21,500 rpm for 1 minute. After centrifugation at 47,500 rpm for 5 minutes, the resulting nanoparticles were separated, obtaining yellow solid powders. The size and the shape of the nanoparticles were confirmed with scanning electron microscopy (SEM) by using QUANTA FEG 250 (FEI company, Hillsboro, Oreg.) of Masdar Institute of Science and Technology (Abu Dhabi, UAE).

### Comparative Example 1-2

100 mg of Zein (Sigma-Aldrich) was dissolved in 1.825 ml of 70% aqueous alcohol solution. 52.5 mg of Sorbitol (Sigma-Aldrich) and 67.5 mg of glycerol formal (Sigma-Aldrich) were added to the solution and mixed until the solution became clear. In order to capsulate an anti-oxidative material with Zein, 100 U/ml of SOD (EC 1.15.1.1., Sigma-Aldrich; 1 U of SOD refers to an activity of processing 1 μmol of superoxide within one minute) was added dropwise to the solution and stirred to obtain a mixed solution (A total of 200 μl of SOD was added). A nanoparticle was prepared by using a phase separation method. Shortly thereafter, 8.0 ml of water was added to the above prepared mixed solution which was then dispersed by homogenization at 21,500 rpm for 1 minute. After centrifugation at 47,500 rpm for 5 minutes, the resulting nanoparticles were separated, obtaining yellow solid powders. The size and the shape of the nanoparticles were confirmed with scanning electron microscopy (SEM) by using QUANTA FEG 250 (FEI company, Hillsboro, Oreg.) of Masdar Institute of Science and Technology (Abu Dhabi, UAE).

### Comparative Example 2

### Preparation of Film for Oral Delivery Containing an Anti-Oxidative Enzyme

### Comparative Example 2-1

450 mg of Zein (Sigma-Aldrich) was dissolved in 1.825 ml of 50% aqueous alcohol solution. 52.5 mg of Sorbitol and 67.5 mg of glycerol formal were added to the solution and mixed until the solution became clear. 3 mg/ml of catalase was added dropwise to the solution and stirred to obtain a mixed solution (A total of 200 μl of catalase was added). A film was prepared by evaporating the mixed solution at an ambient temperature (25° C.) over 24 hours.

### Comparative Example 2-2

450 mg of Zein (Sigma-Aldrich) was dissolved in 1.825 ml of 50% aqueous alcohol solution. 52.5 mg of Sorbitol and 67.5 mg of glycerol formal were added to the solution and mixed until the solution became clear. 100 U/ml of SOD was added dropwise to the solution and stirred to obtain a mixed solution (A total of 200 μl of SOD was added). A film was prepared by evaporating the mixed solution at an ambient temperature (25° C.) over 24 hours.

### Comparative Example 2-3

100 mg of Zein (Sigma-Aldrich) was dissolved in 1.825 ml of 70% aqueous alcohol solution. 67.5 mg of glycerol formal (Sigma-Aldrich) were added to the solution and mixed until the solution became clear. 3 mg/ml of catalase (EC 1.11.16.1, Sigma-Aldrich) was added dropwise to the solution and stirred to obtain a mixed solution (A total of 200 μl of catalase was added). A Zein film was prepared by spreading and drying the mixed solution over a glass container at an ambient temperature (25° C.) over 24 hours.

### Example 1

### Preparation of a Prolamin-Based Film for Oral Delivery Containing an Anti-Oxidative Enzyme in Accordance with the Present Invention

### Example 1-1

### Preparation of Zein-Based Film for Oral Delivery Containing Catalase

500 mg of Zein (Sigma-Aldrich) was dissolved in 10 ml of 70% aqueous alcohol solution. 335 mg of glycerol formal (Sigma-Aldrich) was added to the solution and mixed until the solution became clear. 2 ml of the solution (including 100 mg of Zein) was placed into a reduced pressure flask in which 3 mg/ml of catalase (EC 1.11.16.1, Sigma-Aldrich) was then added dropwise and mixed to obtain a mixed solution (A total of 200 μl of catalase was added). Until a thin film was obtained, a solvent (i.e., ethanol) was evaporated from the mixed solution in the reduced pressure flask by using a rotary evaporator under the reduced pressure of 0.002 mbar at 35° C.

### Example 1-2

### Preparation of Zein-Based Film for Oral Delivery Containing Superoxide Dismutase (SOD)

500 mg of Zein was dissolved in 10 ml of 70% aqueous alcohol solution. 335 mg of glycerol formal was added to the solution and mixed until the solution became clear. 2 ml of the solution (including 100 mg of Zein) was placed into a reduced pressure flask in which 100 U/ml of SOD (EC 1.15.1.1, Sigma-Aldrich; 1 U of SOD refers to an activity of processing 1 μmol of superoxide within one minute) was added dropwise to the solution and stirred to obtain a mixed solution (A total of 200 μl of SOD was added). Until a thin film was obtained, a solvent (i.e., 70% ethanol) was evaporated from the mixed solution in the reduced pressure flask by using a rotary evaporator under the reduced pressure at 35° C.

### Example 1-3

### Preparation of Gliadin-Based Film for Oral Delivery Containing Catalase

500 mg of Gliadin (Sigma-Aldrich) was dissolved in 10 ml of 70% aqueous alcohol solution. 335 mg of glycerol formal (Sigma-Aldrich) was added to the solution and mixed until the solution became clear. 2 ml of the solution (including 100 mg of Gliadin) was placed into a reduced pressure flask in which 3 mg/ml of catalase was then added dropwise and mixed to obtain a mixed solution (A total of 200 μl of catalase is added). Until a thin film was obtained, a solvent (i.e., 70% ethanol) was evaporated from the mixed solution in the reduced pressure flask by using a rotary evaporator under the reduced pressure at 35° C.

### Example 2

### Effect of the Prolamin-Based Film for Oral Delivery According to the Present Invention in Protecting the Activity of an Anti-Oxidative Enzyme

The activities of the anti-oxidative enzymes as contained in the prolamin-based film for oral delivery prepared according to Example 1 were measured as follows:

### Example 2-1

### Prolamin-Based Film for Oral Delivery System

In the prolamin-based film for oral delivery containing catalase, the activity of catalase was measured by checking the decomposition rate of hydrogen peroxide (H2O2). The activity of catalase was measured by mixing catalase or the prolamin-based film for oral delivery containing catalase with 10 μM of H2O2 in a buffering solution of pH 7.0, and then monitoring the absorbance value of H2O2 at 240 nm. Further, in the prolamin-based film for oral delivery containing SOD, SOD activity was measured in accordance with the manufacturer's instructions by using SOD assay kit (SOD determination kit 19,160, Fluka, St. Louis, Mo.). Significance of results was determined via t-test between Zein film and other films containing SOD or catalase with p<0.05(*).

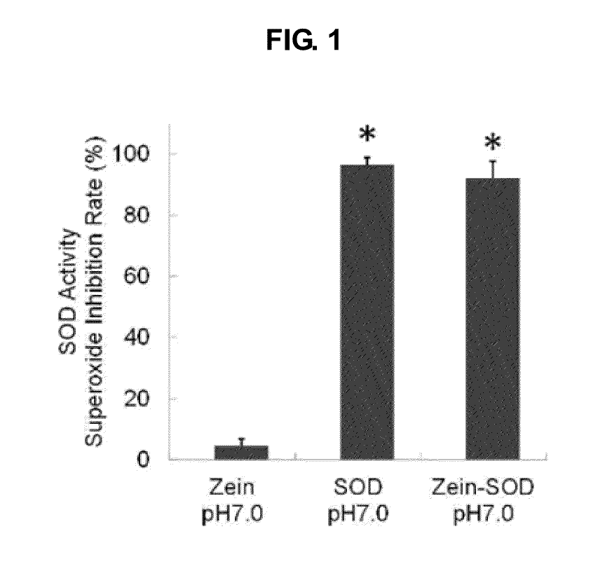

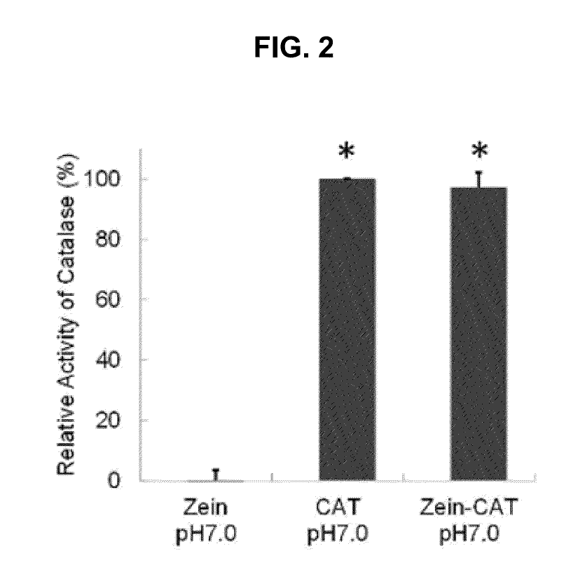

As shown in FIG. 1 and FIG. 2, the activity of either SOD or catalase alone at pH 7.0 was approximately similar to those as contained in the Zein-based film for oral delivery, respectively. These results confirmed that SOD and catalase maintained their activities at the same level even after they were formulated with Zein into a film-based drug delivery system.

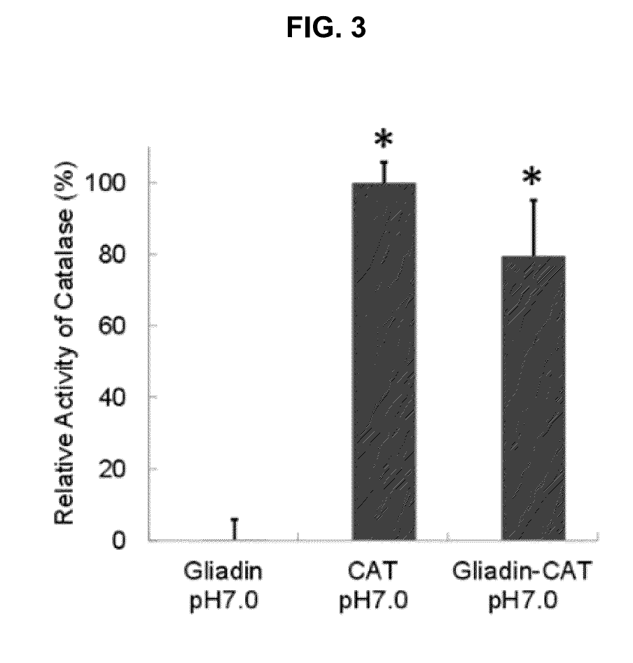

In the same manner, as demonstrated in FIG. 3, it was confirmed that catalase maintained its anti-oxidative activity even after it was contained in the film prepared with Gliadin.

### Example 2-2

### Evaluation of the Activity of an Anti-Oxidative Enzyme in the Stomach Condition

The function of Zein in protecting an anti-oxidative protein in the stomach condition was evaluated. In order to mimic the stomach condition, catalase or the prolamin-based film for oral delivery containing catalase was added into hydrochloric acid solution (50.1 mM HCl, pH 1.3) and left for 20 minutes. Then, test groups were divided into 349.1 mM Pepsin-treated group and Pepsin-untreated group, respectively (See Metheny, N. A., Stewart, B. J., et al., pH and Concentrations of Pepsin and Trypsin in Feeding Tube Aspirates as Predictors of Tube Placement, J. PARENTER. ENTERAL NUTR. 21:5, 279-85 (1997)). The activities of catalase and SOD were measured in the same manner as Example 2-1. Significance of results was determined via t-test between free enzymes and other delivery vehicles with p<0.05(*).

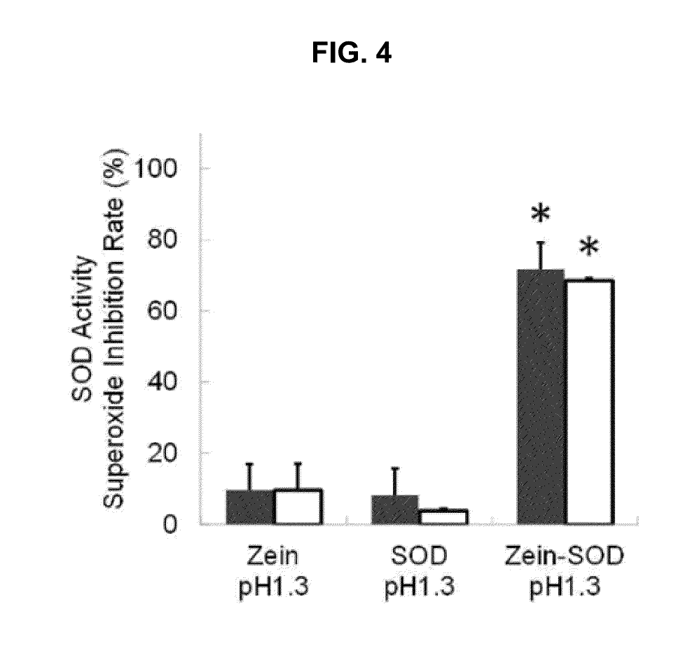

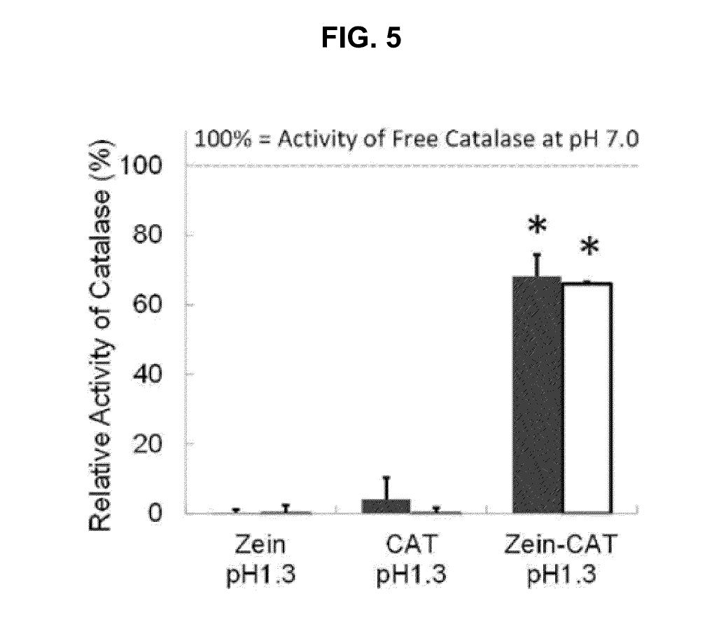

As shown in FIG. 4 and FIG. 5, either SOD or catalase alone lost almost completely its activity in the acidic stomach condition (pH 1.3). On the contrary, it was found that SOD or catalase contained in the Zein-based film maintained the high level of its activity even in the stomach condition (pH 1.3). Further, in either pepsin-treated or pepsin-untreated group, there was no significant difference in the activity of SOD or catalase. Hence, the Zein-based film as prepared according to an exemplary embodiment proved to be excellent in protecting the activity of an anti-oxidative material.

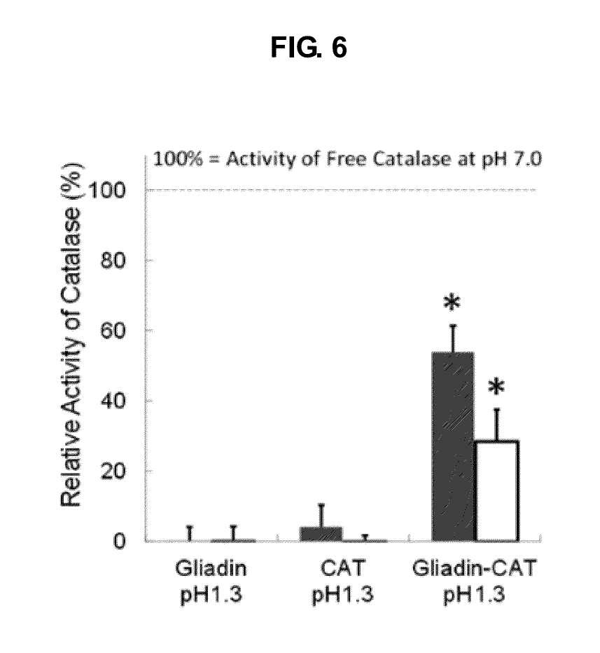

As further shown in FIG. 6, it was confirmed that the anti-oxidative activity of catalase contained in the Gliadin-based film was protected in the stomach condition (pH 1.3).

### Example 2-3

### Evaluation of the Activity of an Anti-Oxidative Enzyme in the Small Intestine Condition

In order to mimic the small intestine condition, test groups were divided into 143.0 mM Trypsin-treated group and Trypsin-untreated group, respectively, while being left for 30 minutes in the environment of pH 7.4.

The activities of catalase and SOD were measured in the same manner as Example 2-1. Significance of results was determined via t-test between free enzymes and other delivery vehicles with p<0.05(*).

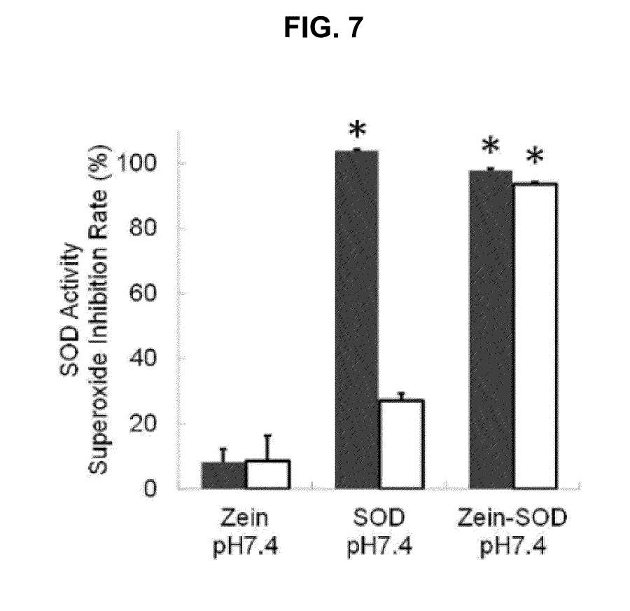

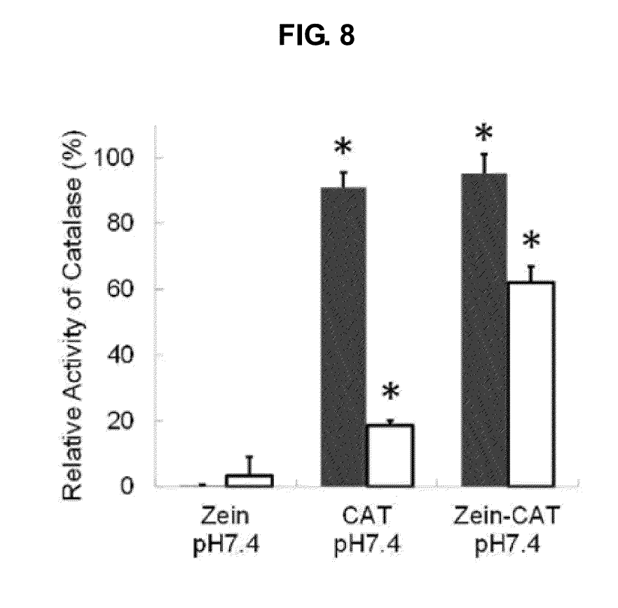

As shown in FIG. 7 and FIG. 8, while either SOD or catalase alone maintained its activity under the condition of pH 7.4, it was found that either SOD or catalase alone lost its activity significantly in the Trypsin-treated group which is similar to the small intestine condition. In contrast, it was confirmed that SOD or catalase contained in the Zein-based film maintained its activity at its high level, respectively, regardless of the treatment of Trypsin.

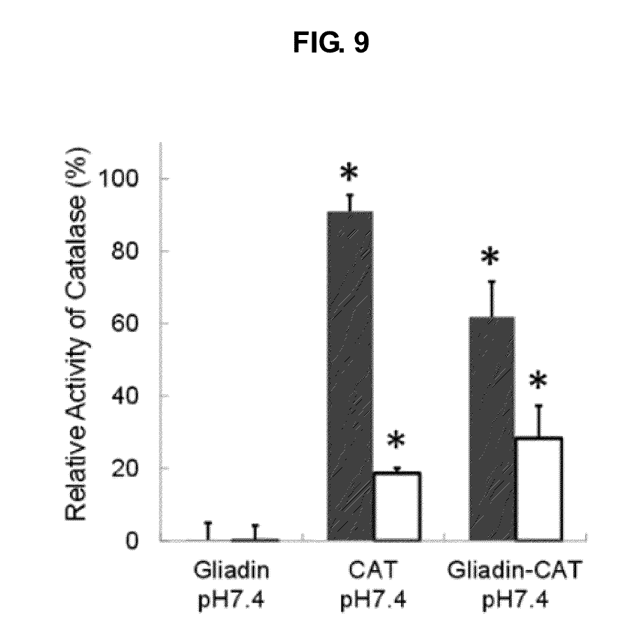

As further shown in FIG. 9, it was confirmed that the anti-oxidative activity of catalase contained in the Gliadin-based film was protected in the small intestine condition (pH 1.3).

### Experimental Example 1

### Activity Assessment Depending on the Type of Formulation

The activities of catalases contained in the film for oral delivery as prepared in the Comparative Example 1-1 and Example 1-1 were compared. First, in order to determine the amount of catalase contained in each formulation, fluorescein isothiocyanate (FITC, Sigma-Aldrich) was chemically bonded to catalase, resulting in the formation of Zein Nanoparticle and Zein Film, respectively. 1 mg of each formulation was taken to check the quantity of fluorescence which was then compared with the initially used quantity of fluorescence, resulting in the determination of the quantity of catalase in each formulation. The activity of catalase was evaluated at pH 7.0 as described in Example 2-1 by using fluorescent-unbound catalase, forming Zein Nanoparticle and Film, and taking 1 mg of each formulation.

TABLE 1
Loading Efficiency of Catalase in Each Formulation

| Catalase in Zein Nanoparticle | Catalase in Zein Film |
|---|---|
| 40.99 ± 4.27% | 98.58 ± 1.23% |

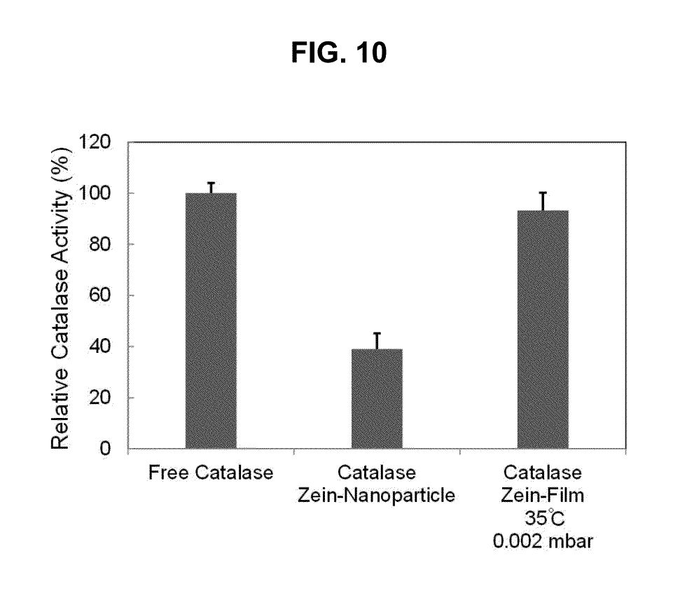

As shown in Table 1 and FIG. 10, it was found that catalase contained in the film for oral delivery maintained its much higher activity than one in the nanoparticle. This result suggests that the film contains a much higher amount of catalase during its preparation process.

### Experimental Example 2

### Activity Assessment Depending on the Type of Preparation Process

### Experimental Example 2-1

### Comparison Between the Conventional Process and the Inventive Process of Preparing the Film for Oral Delivery

1 mg of each film for oral delivery as prepared in Comparative Example 2-3 and Example 1-1 was respectively taken to evaluate the activity of catalase contained therein. The activity assessment of catalase was made at pH 7.0 as described in Example 2-1.

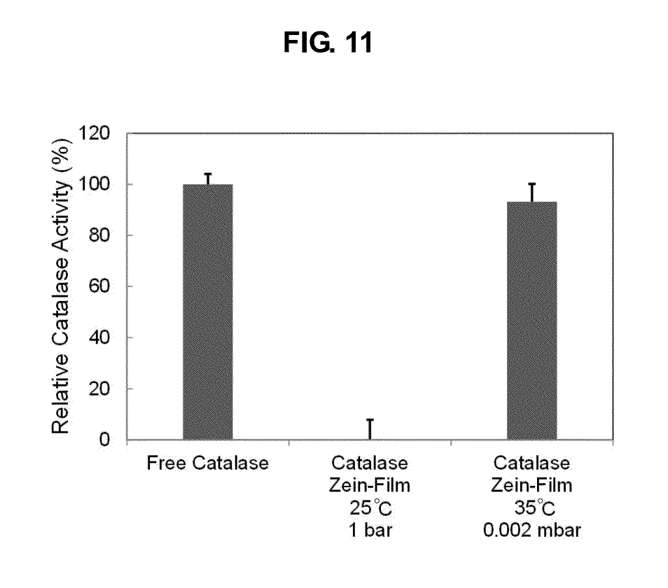

As shown in FIG. 11, it was found that the film for oral delivery as prepared in the conventional process lost the activity of catalase contained therein almost completely, whereas the film as prepared according to an exemplary embodiment maintained the activity of catalase at a significantly high degree.

### Experimental Example 2-2

### Comparison Among the Films for Oral Delivery Prepared at Varied Temperatures

In various films for oral delivery as prepared according to various exemplary embodiments by using varied temperatures at the same reduced pressure of 0.002 mbar, the activities of anti-oxidative enzymes contained therein were evaluated.

Except for pressure and temperature conditions, the same methods as described in Example 1-1 were used to prepare the films for oral delivery.

As shown in FIG. 12 and FIG. 13, catalases contained in the films for oral delivery prepared under the temperature of 0 to 45° C., in particular 15 to 35° C., maintained the relatively high degree of their activities.

### Experimental Example 2-3

### Comparison Among the Films for Oral Delivery Prepared at Varied Pressures

In various films for oral delivery as prepared according to various exemplary embodiment by using varied pressures at the same temperature of 35° C., the activities of anti-oxidative enzymes contained therein were evaluated.

Except for pressure and temperature conditions, the same methods as described in Example 1-1 were used to prepare the films for oral delivery.

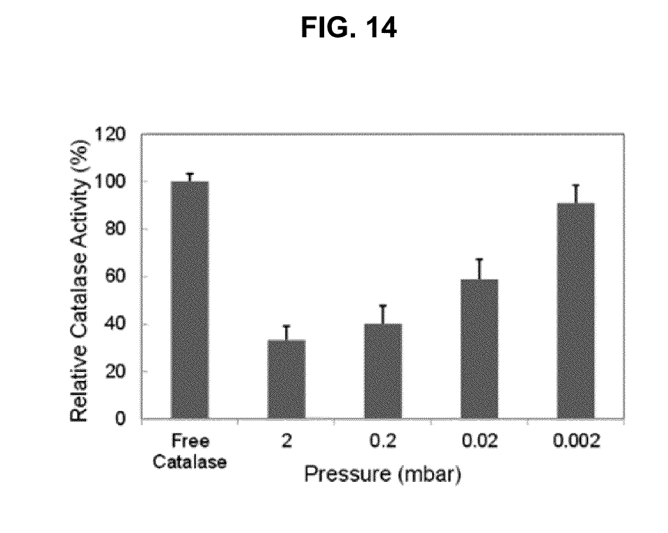

As shown in FIG. 14, it was confirmed that as the lower pressure was applied, the stronger activity of catalases contained in the films for oral delivery were maintained. In particular, the activity of catalase was retained at the very high level at the pressure of 0.002 mbar.

### Example 3

### Prolamin-Based Film for Oral Delivery Containing a Non-Enzymatic Anti-Oxidative Material as Prepared by the Present Invention

The films for oral delivery were prepared as described in Example 1-1 by using 5 mg of caffeine, and 100 mg of Gliadin or Zein as prolamin. In order to assess the potential of prolamin in protecting an anti-oxidative material, caffeine and the prolamin-based film for oral delivery containing caffeine were respectively added into hydrochloric acid solution (50.1 mM HCl, pH 1.3) and left for twenty minutes for the purpose of mimicking the stomach condition. Subsequently, the solution was neutralized to pH 7.0 and used for cellular analysis.

In order to measure the anti-oxidative activity of caffeine as contained in the film for oral delivery, cells (RAW264.7, 4×10⁵ cells/well) were cultured in a 12 well plate. Then, 1 mg of prepared film for oral delivery as described above was cultured with the cells in each well for 2 hours, while the culture solution being removed thereafter. The cells were treated with hydrogen peroxide (H₂O₂) (100 μM) for 4 hours. Subsequently, the viability of cells from the treatment of hydrogen peroxide and the potential of caffeine in protecting cells from hydrogen peroxide were measured.

Test groups were divided as follows: A—Negative Control Group; B—Group treated with H₂O₂ only; C—Group treated with a caffeine solution left at pH 1.3 for 20 minutes, and H₂O₂; D—Group treated with a caffeine solution left at pH 1.3 for 20 minutes followed by neutralization of its pH to 7.0, and H₂O₂; E—Group treated with a solution of the Gliadin-based film for oral delivery containing caffeine left at pH 1.3 for 20 minutes, and H₂O₂; F—Group treated with a solution of the Gliadin-based film for oral delivery containing caffeine at pH 1.3 for 20 minutes followed by neutralization of its pH to 7.0, and H₂O₂; G—Group treated with a solution of the Zein-based film for oral delivery containing caffeine left at pH 1.3 for 20 minutes, and H₂O₂; H—Group treated with a solution of the Zein-based film for oral delivery containing caffeine at pH 1.3 for 20 minutes followed by neutralization of its pH to 7.0, and H₂O₂.

Cytotoxicity was measured by using propidium iodide (PI), a fluorescent material which stains only dead cells. 100 μl of PI (500 μg/ml PI) was added to each well. 10 minutes later, microplate reader manufactured by Tecan was used for measuring a fluorescence(excitation: 535 nm, emission: 617 nm). Relative cytotoxicity in other test samples was described in comparison with the cases of culture solution only or hydrogen peroxide only which were designated as 0% and 100%, respectively.

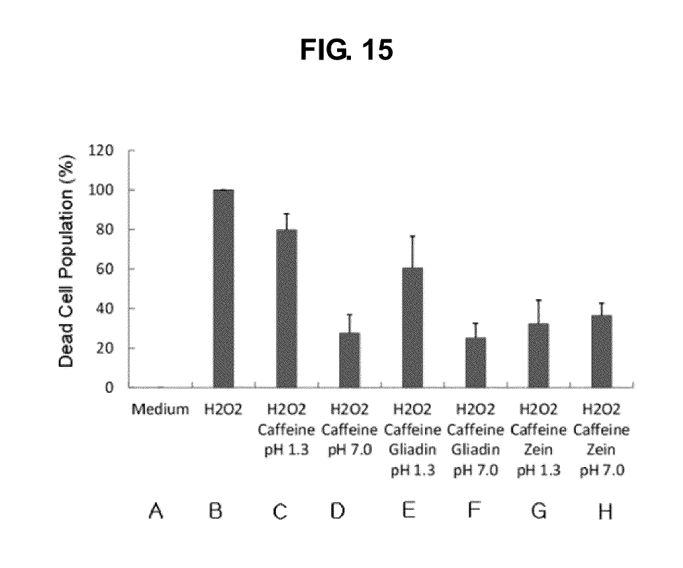

Caffeine is reportedly able to protect cells from reactive oxygens (See Silverberg J. I., et al., Caffeine Protects Human Skin Fibroblasts from Acute Reactive Oxygen Species-Induced Necrosis, J DRUGS DERMATOL. 11:1342-6 (November 2012)). Further, as shown in FIG. 15, the above experiments confirmed that the activity of caffeine contained in Gliadin- and Zein-based film for oral deliveries was maintained well. The above data confirmed that the preparation methods of prolamin (especially, Zein and Gliadin)-based films for oral delivery according to various exemplary embodiments may be applied well to both anti-oxidative enzymes and non-enzymatic anti-oxidative materials.

### Preparation Examples

Preparation examples of the prolamin-based film for oral delivery containing an anti-oxidative material in accordance with various exemplary embodiments are as follows:

### Preparation Example 1

### Gliadin-Based Film for Oral Delivery Containing SOD

Using 100 mg of Gliadin, 67.5 mg of glycerol formal, and 200 μL of SOD, the film for oral delivery was prepared in the same manner as Example 1-2.

### Preparation Example 2

### Hordein-Based Film for Oral Delivery Containing Catalase

Using 100 mg of Hordein, 67.5 mg of glycerol formal, and 200 μL of catalase, the film for oral delivery was prepared in the same manner as Example 1-1.

### Preparation Example 3

### Glutenin-Based Film for Oral Delivery Containing Catalase

Using 100 mg of Glutenin, 67.5 mg of glycerol formal, and 200 μL of catalase, the film for oral delivery was prepared in the same manner as Example 1-1.

### Preparation Example 4

### Secalin-Based Film for Oral Delivery Containing Catalase

Using 100 mg of Secalin, 67.5 mg of glycerol formal, and 200 μL of catalase, the film for oral delivery was prepared in the same manner as Example 1-1.

### Preparation Example 5

### Coixin-Based Film for Oral Delivery Containing SOD

Using 100 mg of Coixin, 67.5 mg of glycerol formal, and 200 μL of SOD, the film for oral delivery was prepared in the same manner as Example 1-2.

### Preparation Example 6

### Zein-Based Film for Oral Delivery Containing Glutathione Peroxidase

Using 100 mg of Zein, 67.5 mg of glycerol formal, and 200 μL of glutathione peroxidase, the film for oral delivery was prepared in the same manner as Example 1-1.

### Preparation Example 7

### Zein-Based Film for Oral Delivery Containing Glutathione Reductase

Using 100 mg of Zein, 67.5 mg of glycerol formal, and 200 μL of glutathione reductase, the film for oral delivery was prepared in the same manner as Example 1-1.

### Preparation Example 8

### Zein-Based Film for Oral Delivery Containing Glutathione-S-Transferase Hemeoxygenase

Using 100 mg of Zein, 67.5 mg of glycerol formal, and 200 μL of glutathione-S-transferase hemeoxygenase, the film for oral delivery was prepared in the same manner as Example 1-1.

### Preparation Example 9

### Zein-Based Film for Oral Delivery Containing Vitamin C

Using 100 mg of Zein, 24 mg of glycerol formal, and 5 mg of vitamin C, the film for oral delivery was prepared in the same manner as Example 3.

### Preparation Example 10

### Zein-Based Film for Oral Delivery Containing Tocopherol

Using 100 mg of Zein, 24 mg of glycerol formal, and 5 mg of tocopherol, the film for oral delivery was prepared in the same manner as Example 3.

As set forth above, the present disclosure relates to a prolamin-based film and a method for manufacturing a prolamin-based film for oral delivery for protecting the activity of an anti-oxidative material. In particular, the present disclosure relates to a method for manufacturing a prolamin-based film for oral delivery, the method including (a) preparing an aqueous alcohol solution containing prolamin, glycerol formal, and an anti-oxidative material and (b) evaporating a solvent under a reduced pressure condition at a temperature of 0° C. to 80° C. According to various exemplary embodiments, a film is prepared for oral delivery containing an anti-oxidative material that maintains a high level of activity that is retained at a high level even under the harsh conditions of the stomach and the small intestine, rendering the film for oral delivery highly industrially applicable.

What is claimed is:

1. A method for preparing a film for oral delivery, the method comprising:
(a) preparing a solution comprising prolamin, glycerol formal, an anti-oxidative material, and a solvent comprising alcohol; and
(b) evaporating the solvent under a reduced pressure condition at a temperature of 0° C. to 80° C.

2. The method of claim 1, wherein the prolamin comprises at least one plant source-derived prolamin selected from the group consisting of wheat, rye, barley, oat, rice, sorghum, millet, coix, and maize.

3. The method of claim 2, wherein the prolamin comprises at least one selected from the group consisting of Zein, gliadin, hordein, secalin, kafirin, coixin, glutenin, GluB-Glutelin, and avenin.

4. The method of claim 1, wherein the anti-oxidative material comprises at least one selected from the group consisting of an anti-oxidative enzyme, a non-enzymatic anti-oxidative material, and a mixture thereof.

5. The method of claim 4, wherein the anti-oxidative material is an anti-oxidative enzyme and the anti-oxidative enzyme comprises at least one selected from the group consisting of superoxide dismutase, catalase, glutathione peroxidase, glutathione reductase, glutathione-S-transferase hemeoxygenase, a synthetic enzyme thereof, and a peptide mimetic thereof.

6. The method of claim 4, wherein the anti-oxidative material comprises a non-enzymatic anti-oxidative material and the non-enzymatic anti-oxidative material comprises at least one selected from the group consisting of vitamin E, curcumin, carotene, lipoic acid, Coenzyme Q10, tocopherol, polyphenol, retinyl palmitate, thiotic acid, carotenoid, vitamin C, vitamin C derivatives, caffeine, caffeic acid, butylated hydroxyanisole (BHA), butylated hydroxytoluene (BHT), tertiary butylhydroquinone (TBHQ), epigallocatechin-3-gallate (EGCG), proanthocyanidin, gallic acid, selenium, zinc, taurine, flavonoid, and scopoletin.

7. The method of claim 1, wherein the temperature is in the range of 0° C. to 45° C.

8. The method of claim 1, wherein the temperature is in the range of 10° C. to 38° C.

9. The method of claim 1, wherein the pressure is in the range of 1×10⁻⁵ mbar to 999 mbar.

10. The method of claim 1, wherein step (a) further comprises:
(i) dissolving prolamin in the solvent to form a first solution;
(ii) adding the glycerol formal to the first solution of step (i) to form a second solution; and
(iii) adding the anti-oxidative material to the second solution of step (ii).

11. The method of claim 1, wherein the solvent further comprises water.

12. A prolamin-based film for oral delivery comprising an anti-oxidative material prepared according to the method of claim 1.

13. A film, comprising:
prolamin;
glycerol formal; and
an anti-oxidative material having a high level of activity.

14. The film of claim 13, wherein the prolamin comprises at least one plant source-derived prolamin selected from the group consisting of wheat, rye, barley, oat, rice, sorghum, millet, coix, and maize.

15. The film of claim 14, wherein the prolamin comprises at least one selected from the group consisting of Zein, gliadin, hordein, secalin, kafirin, coixin, glutenin, GluB-Glutelin, and avenin.

16. The film of claim 13, wherein the anti-oxidative material comprises at least one selected from the group consisting of an anti-oxidative enzyme, a non-enzymatic anti-oxidative material, and a mixture thereof.

17. The film of claim 16, wherein the anti-oxidative material comprises an anti-oxidative enzyme and the anti-oxidative enzyme comprises at least one selected from the group consisting of superoxide dismutase, catalase, glutathione peroxidase, glutathione reductase, glutathione-S-transferase hemeoxygenase, a synthetic enzyme thereof, and a peptide mimetic thereof.

18. The film of claim 16, wherein the anti-oxidative material comprises an non-enzymatic anti-oxidative material and the non-enzymatic anti-oxidative material comprises at least one selected from the group consisting of vitamin E, curcumin, carotene, lipoic acid, Coenzyme Q10, tocopherol, polyphenol, retinyl palmitate, thiotic acid, carotenoid, vitamin C, vitamin C derivatives, caffeine, caffeic acid, butylated hydroxyanisole (BHA), butylated hydroxytoluene (BHT), tertiary butylhydroquinone (TBHQ), epigallocatechin-3-gallate (EGCG), proanthocyanidin, gallic acid, selenium, zinc, taurine, flavonoid, and scopoletin.

19. The film of claim 13, further comprising an alcohol.

20. The film of claim 19, further comprising water, wherein the alcohol is ethanol.
